(19) United States
(12) Patent Application Publication
(10) Pub. No.: US 2003/0172409 A1
(43) Pub. Date: Sep. 11, 2003

(54) Methods for increasing biomass of transgenic maize

(75) Inventor: Michael E. Horn, College Station, TX (US)

Correspondence Address: MCKEE, VOORHEES & SEASE, P.L.C., 801 GRAND AVENUE, SUITE 3200, DES MOINES, IA, 50309-2721, US

(73) Assignee: Prodigene, Inc., College Station, TX

(21) Appl. No.: 10/348,700

(22) Filed: Jan. 22, 2003

Related U.S. Application Data
(60) Provisional application No. 60/350,451, filed on Jan. 22, 2002.

(51) Int. Cl. A01H 1/00; C12N 15/82; A01H 5/00

(52) U.S. Cl. 800/294; 800/288; 800/320.1

(57) ABSTRACT

A method of improving seed set of transgenic maize plants and improving agronomic characteristics of such plants is disclosed which employs the use of maize host cells that are the result of a cross between one parent that is Stiff Stalk derived germplasm and a second parent that is from the HiII genotype.

### BACKGROUND OF THE INVENTION

A major goal in the new area of Molecular Farming is increased speed to adequate quantities of any protein product. Quantities of 10-100 mg are needed for protein characterization and/or small-scale pre-clinical studies. Protein quantities of 10-20 g are needed for pre-clinical toxicity tests and 50-100 g are needed for Phase I clinical studies. Any procedure that shortens the path between having the gene construct and obtaining the necessary quantity of transprotein can mean the difference between success and failure in the molecular farming industry. This is because potential clients need to put the potential protein products into clinical testing in the shortest time possible.

Plants make excellent protein factories. Corn in particular makes protein less expensively than can be done using CHO cells or other bioreactor systems. A bushel of corn seed (˜25 kg) containing 0.01% of its total dry weight in transprotein (2.5 g) can be grown for about $2.50-3.00. An average of 200 bushels per acre is a reasonable estimate and thus, using the 0.01% figure from above, just one acre can produce 500 g of a desired transprotein minus any loss due to extraction and purification. Levels of transprotein exceeding 0.1% total dry weight have now been recorded in corn seed but the actual level must be determined for each protein.

Significant progress has been made in the last 10 years to make maize transformation less difficult. The use of Agrobacterium tumefaciens was one such advance (Ishida et al., 1996) and is now routinely practiced in a number of laboratories. The advantages of A. tumefaciens as a transformation vector are many but the most important of these is the scarcity of multi-copy transgenic events. Single copy events have far fewer regulatory hurdles in the path to commercialization. One drawback in using A. tumefaciens for corn transformation, however, is the relatively small number of corn genotypes that are amenable to both Agrobacterium infection and tissue culture practices. One genotype commonly used for all transformation systems is HiII (Armstrong et al., 1991). Why this particular genotype works when most others do not is still a matter of speculation (Armstrong et al., 1992). While HiII is a very effective transformation host and performs very well in culture, it has serious agronomic problems in the greenhouse and the field. Seed set on HiII T₀ plants in a Texas greenhouse in summer can be minimal and pollen shed can be severely affected as well.

### SUMMARY OF THE INVENTION

The invention comprises a method of improving Agrobacterium mediated maize transformation as well as agronomic characteristics of transformed plants that includes providing for transformation a plant cell, the cell being the result of a cross between a first parent which is derived from a Stiff Stalk germplasm pool and a second parent which is a HiII genotype, and contacting said the progeny tissue with an Agrobacterium based vector under transformation conditions. There may be further crosses of the progeny of the first and second parent with the same or a different Stiff Stalk germplasm plant prior to transformation.

### DESCRIPTION OF THE FIGURES

FIG. 1 is a picture of HiIIxSP122 callused embryos compared to HiII callused embryos two weeks after treatment with Agrobacterium carrying the PGN9048 construct.

FIG. 2 is a picture of L-R SP122 from seed, SP122 x HiII T₀ plant, HiII T₀ plant, HiII from seed. The tallest plant is about 2.13 m.

FIG. 3 is a picture of a typical mature T₁ ear from HiII (L) compared to a typical mature T₁ ear from HiIIxSP122 (R).

### DETAILED DESCRIPTION OF THE INVENTION

The transformation of elite maize genotypes continues to be a difficult exercise even using techniques such as particle bombardment (O'Kennedy et al., 2001). Low efficiencies and the high potential for multiple gene copies make particle bombardment a challenging method for elite transformation in our field of molecular farming in which regulatory concerns are always a factor. Agrobacterium-based transformation of maize leads to single copy insertions in 90%+ of the transgenic events recovered. However, Agrobacterium is quite genotype specific in its host range in many agronomically important species and maize is no exception. Transient GUS assays have shown poor gene transfer when using elite maize immature embryos (data not shown). HiII/Elite embryos were hypothesized to be a possible good compromise in that the transformability/culturability ability from the HiII parent might allow some reasonable level of transformation to occur. A transformation frequency was expected of half or less of that seen with HiII. To our surprise, transformation efficiencies using certain Stiff Stalk elite inbreds as one parent showed levels no different from that of HiII alone. This implies that those genes responsible for transformability in HiII can be dominant in at least some crosses. Lancaster types, while capable of working at a low frequency in at least one case (SP114xHiII), do not seem to have the propensity for high efficiency transformation. A hypothesis that using HiII as the female parent might provide superior results also did not prove true. In fact, the direction of the HiII/Elite cross did not matter at all. Subsequently, when shifting commercial production to HiIIxSP122 immature embryos and transformation frequencies as high as 10.3% have been observed.

To boost T₁ seed set and speed up the product development path, the inventors examined the possibility of using A. tumefaciens transformation on hybrid embryos derived from a cross between HiII and elite germplasm. The results were surprising in the magnitude of the system improvements. In addition, it was surprising that transformation was mostly limited to crosses between HiII and Stiff Stalk elite inbreds. Seed set was indeed boosted dramatically as was the transprotein level in the T₁ seed. Hybrid embryos resulting from crosses between a highly regenerable maize germplasm (HiII) and certain elite inbreds were treated with Agrobacterium tumefaciens containing the GUS and pat genes under the control of two different constitutive promoters, respectively. The elite inbred lines consisted of three Lancaster and three Stiff Stalk types. Hybrid embryos from all three Stiff Stalk lines gave transgenic events at various frequencies; two not significantly lower than with HiII embryos. Only one Lancaster type showed successful transformation as part of a hybrid with HiII and the frequency was quite low. The resultant transgenic events showed many characteristics of the elite inbred parent including more aggressive rooting, thicker stems, and taller stature than plants derived from HiII events. The hybrid T₀ plants also exhibited excellent tassel development in the greenhouse with abundant pollen shed. Seed set in the greenhouse was significantly (3-4 fold) higher than with HiII transformants. Attempts to transform embryos derived from self or sibling crosses of the four inbred lines successful as hybrids with HiII did not produce any transgenic events. Nevertheless, T₀ plants having 50% elite genomic contribution perform nearly as well in the greenhouse as seed-derived elite parents and offer a significantly reduced time line for transprotein product development. Only a modest increase in seed set using hybrid embryos was predicted, but the magnitude of the improvements throughout the entire system was surprising. These results show that a 3-4 fold increase in seed set and lower incidence of sterility in the HiII/Elite system will always result in a larger pool from which to select the high-expressing individuals. We look for these individuals in order to establish high-expressing parental lines. Of perhaps even more importance, it is possible with this invention to produce 10-30 mg quantities of transprotein in the T₁ seed generation from the greenhouse assuming minimum expression levels are achieved. One could not consider doing this with HiII embryos as the starting material because the seed yields were always inadequate.

The genotype HiII (Armstrong et al., 1991), while having high culturability and transformability traits, is not a robust genotype and does not tolerate temperature extremes well. Nor does HiII have good agronomic characteristics in the field. Elite genotypes, on the other hand, have historically been recalcitrant in culture. Even those elite lines that do show high Type II callus formation and good plant recovery absent herbicide selection (Duncan et al. 1985), do not usually exhibit good transformation frequencies and/or good regeneration frequencies following selection (Horn, personal observation). The term elite characterizes a plant or variety possessing favorable agronomic traits, such as, but not limited to, high yield, good grain quality and disease resistance. The term also characterizes parents giving rise to such plants or varieties.

Most modern maize hybrids are crosses between two elite inbreds. These elite inbreds are commonly derived from germplasm pools known as Stiff Stalk and Lancaster. Stiff Stalk inbreds are reported by the USDA to have been first released in 1993. They are derived from the Iowa Stiff Stalk synthetic population. Sprague, G. F. “Early testing of inbred lines of maize” J Amer. Soc. Agron. (1946) 38:108-117. (For example see P1 accession no. 550481; and discussions of Stiff Stalk germplasm at U.S. Pat. Nos. 5,706,603; 6,252,148; 6,245,975; 6,344,599; 5,134,074; and Neuhausen, S. “A survey of Iowa Stiff Stalk parents derived inbreds and BSSS(HT)C5 using RFLP analysis” MNL (1989) 63:110-111). Lancaster inbreds are derived from the open pollinated variety Lancaster Surecrop (See for example, PI 280061). Anderson, E. Sources of effective germplasm in hybrid maize. Annals of the Missouri Botanical Garden (1944) 31:355-361.

A low transformation frequency was initially postulated by the inventors in using HiII/Elite zygotic embryos as starting material because of the genetic contribution from HiII. If successful, the resultant T₀ plants would perhaps give superior seed set relative to HiII alone. They also surmised that hybrids in the HiII x Elite direction (HiII being female) would transform better than the reverse since the cytoplasmic genetic content of HiII might contain elements that help HiII perform so well in culture.

Corn immature embryos were isolated from greenhouse grown ears at 9-13 days after pollination depending on embryo size, generally 1.5-2.0 mm long. The embryos were treated with A. tumefaciens, strain EHA101, containing either a GUS gene or a gene coding for wild type aprotinin. Transformation of plants using the GUS gene or aprotinin gene can be found in U.S. Pat. Nos. 5,804,694 and 5,767,379 respectively. Transformation methods used herein are also discussed at U.S. Pat. No. 6,087,558. all patents and references cited herein are incorporated herein by reference.

The GUS gene (uidA from E. coli; Jefferson 1987) was driven by a maize ubiquitin-like promoter, and terminated by the pinII terminator (An, G. et al. Plant Cell (1989) 1:115-122). The aprotinin gene was driven by a maize globulinl promoter (Belanger, F. C & Kriz A. L, Genetics (1991)129:863-972, linked to a barley alpha amylase signal sequence (BAASS; Rogers, Biochem (1985) 260:3731-3738) and was terminated by the pinII terminator. Both constructs were attached to the 5′ end of a 35S-pat-35S plant transcription unit (PTU). The embryos were plated onto callus induction medium and incubated in the dark at 19° C. for four days. The embryos were then transferred to callus maintenance medium containing 100 mg/L carbenicillin. Three days later the embryos were transferred to the same medium, but now also containing 5 μM bialaphos, and cultured in the dark at 28° C. They were transferred every two weeks to fresh callus maintenance medium. The callused embryos turned brown and ceased growing after about two weeks on bialaphos. Transgenic calli appeared as early as five weeks following treatment but the majority of events appeared at seven or nine weeks after treatment. The transgenic calli were easily spotted due to their white to pale yellow color, Type II callus phenotype, and rapid growth rate.

The transgenic events were grown for approximately four more weeks and then plated onto regeneration medium in the dark at 28° C. for somatic embryo production. The somatic embryos were removed after three weeks and plated onto germination medium in the light at 25 embryos per plate at 28° C. The embryos germinated after 7-21 days and the plantlets were moved into tubes containing 40 ml of MS minimal medium and left in the light for at least one week for further shoot and root development. The plants were then transferred into soil and left in a high humidity environment for one week before moving to the greenhouse floor.

The results showed that HiII/Elite hybrid embryos can perform as well as HiII embryos with regard to transformation frequency, and better than HiII in virtually all other aspects. However, only Stiff Stalk varieties, when crossed with HiII, gave transformation frequencies comparable to those seen with HiII embryos alone (Table 1). Lancaster types performed quite poorly (SP114-see Plant Variety Protection Certificate 970078) or not at all. Our three initial Lancaster selections contained two with similar pedigrees (SP116 (PVP 9400036) and another selection, the latter not shown). We subsequently tested another three Lancaster types (SP111, (PVP 8700213) SP115, (PVP 9900007) and SP127 (PVP 9000050)). More than 1000 zygotic embryos of these genotypes were treated and no transgenic events were obtained. Some variations within groups of maize must always be expected, as judged by the less desirable showing of SP117 (PVP 9900134), a Stiff Stalk type (Table 1). However, these results show that in selecting a group of maize to use for transformation, one can consistently expect far superior results when using plant tissue from Stiff Stalk germplasm as opposed to Lancaster germplasm.

TABLE 1
A comparison of HiII/Elite combinations with HiII selfs including transformation efficiency and
days to event appearance.

| | #ZEs | #Days to | # Stable | Trans | | #ZEs | #Days to | # Stable | Trans |
|---|---|---|---|---|---|---|---|---|---|
| | Treated | Events | Events | Freq | Treated | Events | | Events | Freq |
| Stiff Stalk inbreds | | | | | Lancaster Inbreds | | | | |
| HiII × SP122 | 1164 | 70 | 7 | 0.60% | HiII × SP114 | 988 | | 0 | 0.00% |
| SP122 × HiII | 1085 | 70 | 4 | 0.37% | SP114 × HiII | 1183 | 77 | 4 | 0.34% |
| HiII × SP220 | 1129 | 71 | 8 | 0.71% | HiII × SP116 | 1006 | | 0 | 0.00% |
| SP220 × HiII | 1158 | 63 | 5 | 0.43% | SP116 × HiII | 937 | | 0 | 0.00% |
| HiII × SP117 | 927 | 91 | 2 | 0.22% | HiII × SP111 | 1015 | | 0 | 0.00% |
| SP117 × HiII | 990 | 91 | 1 | 0.10% | SP111 × HiII | 929 | | 0 | 0.00% |
| | | | | | HiII × SP116 | 1449 | | 0 | 0.00% |
| | | | | | SP116 × HiII | 1065 | | 0 | 0.00% |
| HiII Average | 250103 | 46 | 2190 | 0.88% | | | | | |
| S.D | | 10.2 | | | HiII × SP127 | 1059 | | 0 | 0.00% |
| Range | | 34 to 81 | | | SP127 × HiII | 622 | | 0 | 0.00% |

A more direct comparison was performed between HiII (APF) and SP122 (Stiff Stalk—PVP 200000122) x HiII (APX) embryos using one commercial construct, Globulin1::Aprotinin::BAASS (PGN9048). In this particular experiment, we found a small but insignificant difference in transformation frequency (1.44 vs 1.66%) and no difference in days to event appearance (Table 2). This result shows that in a real world setting, transformation with HiII/elite embryos did not sacrifice either efficiency or time.

TABLE 2
A comparison of HiII and SP122 × HiII embryo transformation

| | | Days | | Trans- |
|---|---|---|---|---|
| | # ZEs | to Event | # Stable | formation |
| Construct | Treated | Appearance | Events | Frequency |
| PGN9048 SP122 × HiII | 1250 | 35 | 18 | 1.44% |
| PGN9048 HiII | 1267 | 35 | 21 | 1.66% |

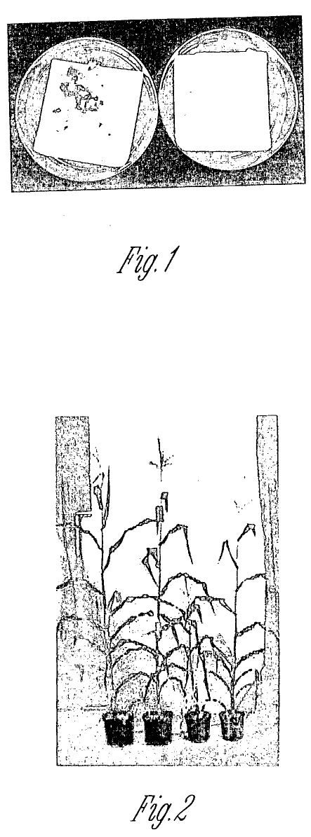

The hybrid zygotic embryos produced far more callus than did HiII embryos in the 2-3 weeks after treatment, before the bialaphos inhibition halts growth (FIG. 1). This may explain the delay we sometimes see in transgenic event appearance in the hybrid material. We hypothesize there is competition for nutrients which slows transformed cells' growth until the non-transgenic calli senesce. Once the hybrid stable events appeared, they exhibited a growth rate equal to or superior to that shown by HiII events.

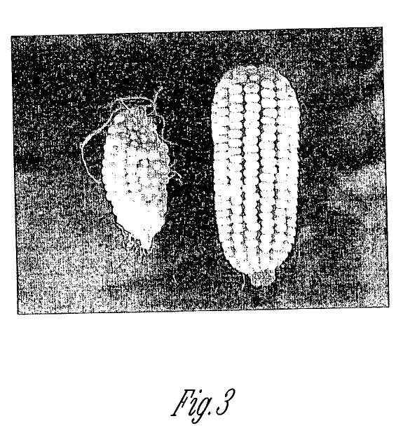

Following a period of tissue growth, the calli were plated onto a medium that induces somatic embryo production. The frequency of somatic embryo production was similarly high in both HiII and HiII/Elite transgenic events (Table 3) but the subsequent germination frequency was much higher with the hybrid somatic embryos compared to the HiII embryos (data not shown). In many plates all 25 somatic embryos germinated to form strong healthy plants. These observations are consistent with the concept of ‘hybrid vigor’ in culture, a phenomenon observed many times by other researchers. These plants performed very well during transition to soil and the greenhouse, forming excellent root systems very quickly in contrast to HiII T₀ plants where the root growth is mediocre at best. The vigor of the HiII/Elite T₀ plants continued into later stages of growth. Mature T₀ plants showed height, stem diameter, anther branching, and pollen shed closely resembling that of seed-derived plants of the same elite genotypes (Table 4, FIG. 2). HiII/Elite T₀ plants have about a 20% increase in stem diameter compared to HiII T₀ plants (4.2 vs. 3.5 cm, respectively). This translated into HiII/Elite T₀ plants that were nearly as tall as the elite parents when the latter was grown from seed in the greenhouse (FIG. 2). Time from planting until flowering was slightly less in the HiII/Elite T₀ plants compared to seed-derived elite parents (54 vs 56-65d, depending on elite parent) but this was not a significant difference. More important are the flowering characteristics. HiII T₀ plants are notorious for male and female sterility, poor tassel development resulting in poor pollen shed, and frequent tassel ear appearance. We found that HiII/Elite T₀ plants showed almost no examples of these problems. Instead, they showed virtually no sterility, excellent tassel development with normal branching and heavy pollen shed (FIG. 2). Consequently, the very important seed yield was exceptionally good with the HiII/Elite T₀ plants. Our test construct, PGN7583, (the gus construct) showed a 3-fold increase in mean seed set compared to PGN7583-transformed HiII T₀ plants (184 vs. 59; Table 3). HiII T₀ plants that do not set any seed are commonplace especially in the heat of the summer months in greenhouses. Barren HiII/Elite T₀ plants have been extremely rare, only four such examples out of 292 HiII/Elite T₀ plants transformed with the PGN7583 construct (1.4%).

TABLE 3
Somatic embryos were plated onto germination medium. Germination
was scored when both a root and a viable shoot had developed.

| | | | Germination |
|---|---|---|---|
| Source | # SEs Plated | # Germinated | Frequency % |
| HiII | 269 | 76 | 29.1 |
| HiII/elite | 400 | 190 | 47.5** |

**Significant difference at the 1% probability level

The comparison using PGN9048 containing the aprotinin gene is a more telling example. There were only eight barren HiII x SP122 T₀ plants out of 357 sent to the greenhouse or 2.2%. This material also averaged 148.5 seeds per ear (Table 4). In contrast, PGN9048-transformed HiII T₀ plants exhibited 45 barren T₀ plants out of 150 sent to the greenhouse (30%) and averaged only 35.3 seeds per ear; 45.7 seeds per ear on those ears that had seed. This calculates to a 4.2-fold increase in seed yield due to the SP122 contribution (FIG. 3).

TABLE 4
T₀ plant characteristics from HiII/SP122 and HiII transgenic events. HiII and HiII/SP122 plants were
pollinated by nontransgenic SP122 pollen. F/S = # fertile plants/# sterile plants;
B/U = # plants with branched tassels/# plants with unbranched tassels;
TE = # plants with tassel ears/# plants with no tassel ears.

| | Zygotic Embryo | Avg # days to | Tassel Development | | | Avg Stalk | Avg # | # Seeds |
|---|---|---|---|---|---|---|---|---|
| Event # | Source | Female Flower | F/S | B/U | TE | Diameter (cm) | Seeds | (Range) |
| 1 | SP122 × HiII | 62.5 | 10/0 | 6/4 | 2/8 | 3.18 | 104 | 7-198 |
| 2 | SP122 × HiII | 61.7 | 10/0 | 10/0 | 0/10 | 5.81 | 249 | 91-375 |
| 3 | SP122 × HiII | 59.0 | 10/0 | 10/0 | 1/9 | 4.08 | 125 | 55-178 |
| 4 | SP122 × HiII | 55.8 | 10/0 | 10/0 | 1/9 | 4.13 | 298 | 225-391 |
| 5 | HiII × SP122 | 64.2 | 8/0 | 8/0 | 1/7 | 5.07 | 235 | 158-325 |
| 6 | HiII × SP122 | 64.7 | 10/0 | 9/1 | 1/9 | 4.08 | 229 | 145-378 |
| 7 | HiII × SP122 | 65.5 | 10/0 | 10/0 | 0/10 | 4.13 | 276 | 184-369 |
| 8 | HiII × SP122 | 55.4 | 10/0 | 10/0 | 1/9 | 4.79 | 284 | 230-368 |
| 9 | HiII × SP122 | 56.1 | 10/0 | 10/0 | 0/10 | 4.63 | 243 | 100-335 |
| 10 | HiII × SP122 | 55.9 | 10/0 | 10/0 | 1/9 | 4.62 | 208 | 53-299 |
| 11 | HiII × SP122 | 52.8 | 10/0 | 9/1 | 0/10 | 4.37 | 252 | 122-364 |
| | | 59.4 | | | | 4.44 | 227 | |
| Control | HiII | 54.4 | 9/1 | 2/8 | 6/4 | 3.77 | 54 | 10-88 |

TABLE 5
A comparison of HiII and HiII × SP122 T₀ plants for fertility, seed set, and aprotinin expression in
the T₁ seed generation.

| | | No. Plants | No. Plants | Avg. Seed | | Avg. Aprotinin | |
|---|---|---|---|---|---|---|---|
| Construct | No. Events | To GH | Setting Seed | Per Ear | s.d. | Yield % TSP* | s.d.* |
| PGN9048 SP122 × HiII | 18 | 372 | 349 | 152 | 81 | 3.09 | 0.92 |
| PGN9048 HiII | 21 | 164 | 105 | 46 | 54 | 2.25 | 1.51 |

*Based on the top 40 ear samples

The most important two qualities of transgenic corn when producing transproteins of pharmaceutical or industrial use is T₁ seed set and transprotein expression level in those seeds. The T₁ seeds from the HiII/Elite T₀ plants contained two doses of the elite germplasm and one dose of HiII, i.e. (HiII/Elite) x Elite. In some cases, the pollen came from the same elite genotype (wild type) and in other cases, the pollen came from an elite plant from the opposite group. For example, a HiII x SP122 T₀ plant might be pollinated with SP122 wt pollen making a (HiII x SP122) x SP122 pedigree. Alternatively, the same T0 plant might be pollinated with pollen from a Lancaster-type elite such as SP114 making the T₁ seed (HiII x SP122) x SP144. Statistical analysis showed there to be no significant difference with regards to seed set or transgene expression and so the data shown in Table 5 is not segregated by pollen parent source.

Table 5 shows that the T₁ seed from the HiII x SP122 transformants expressed 37% more aprotinin than did the seed from the HiII transformants when the top 40 aprotinin-expressing ears in each case were compared. There were 207 more transgenic ears from which to find the 40 highest expressing ears. This fact is a testament to the superior vigor of the T₀ plants, which had a much higher rate of survival from lab to soil in the growth chamber and subsequent establishment onto the greenhouse floor.

Transformation of those elite inbreds that had been successful in a hybrid state with HiII was attempted. A low transformation frequency was believed possible even without the HiII contribution. One thousand immature somatic embryos of SP122, SP220, SP117 (all Stiff Stalk) and of SP114 (Lancaster) were treated with PGN7583 as described above. Although all the embryos were capable of producing Type II callus, no events were recovered. Obviously, the HiII (or HiII-like) genetic contribution is essential for any Agrobacterium-based transformation to occur.

Lastly, attempts were made to transform other Lancaster elite inbreds as hybrids with HiII. Since SP114, a Lancaster type, had given a low transformation frequency and since we observed a wide variation in transformation frequencies within the Stiff Stalk group, it was thought that other Lancaster types might work better than SP114. 1000 immature zygotic embryos of SP111 xHiII, HiIIxSP111, SP115xHiII, HiIIxSP115, SP127xHiII, and HiIIxSP127 were treated with PGN7583 as described above. Again, no transgenic events were observed despite normal Type II callus development.

### REFERENCES

Ishida, Y.; Saito, H.; Ohta, S.; Hiei, Y.; Komari, T.; Kumashiro, T. High efficiency transformation of maize (Zea mays L.) mediated by Agrobacterium tumefaciens. Nat. Biotechnol. 14:745-750; 1996.

Streatfield, S. J.; Jilka, J. M.; Hood, E. E.; Turner, D. D.; Bailey, M. R.; Mayor, J. M.; Woodard, S. L.; Beifuss, K.; Horn, M. E.; Delaney, D. E.; Tizard, I. R.; Howard, J. A. Plant-based vaccines: unique advantages. Vaccine 19: 2742-2748; 2001.

Armstrong, C.; Green, C.; Phillips, R. Development and availability of germplasm with high Type II culture response. Maize Genet. Coop. News Lett. 65:92-93; 1991 Duncan, D. R.; Williams, B. E.; Zehr, B. E.; Widholm, J. M. The production of callus capable of plant regeneration from immature embryos of numerous Zea mays genotypes. Planta 165:322-332; 1985.

Jefferson, R. A.; Kavanaugh, T. A., Bevan, M. W. EMBO J. 6:3901-3907; 1987 Armstrong, C. L.; Romero-Severson, J.; Hodges, T. K. Improved tissue culture response of an elite maize inbred through backcross breeding, and identification of chromosomal regions important for regeneration by RFLP analysis. Theor. Appl. Genet. 84:755-762, 1992.

What is claimed is:

1. A method of Agrobacterium mediated maize transformation comprising:
providing for transformation a hybrid plant cell, said cell being the result of a cross between a first parent which is derived from a Stiff Stalk germplasm pool and a second parent, and
contacting said hybrid embryo with an Agrobacterium based vector under transformation conditions.

2. The method of claim 1 wherein said first parent is the female parent.

3. The method of claim 1 wherein said first parent is the male parent.

4. The method of claim 1 further comprising producing progeny from the cross between the first parent and second parent and crossing the progeny with a plant derived from a Stiff Stalk germplasm pool.

5. The method of claim 1 wherein said Agrobacterium based vector comprises a heterologous nucleotide sequence, the presence of which is desired in a maize cell.

6. The method of claim 1 wherein said second parent is a Hi II genotype.

7. A method of a producing heterologous protein in a maize plant cell comprising:
providing a hybrid maize cell for transformation, and thereafter
introducing to said maize cell an Agrobacterium-based vector comprising a nucleotide sequence encoding said heterologous protein.

8. The method of claim 7 further comprising the step of: harvesting said heterologous protein.

9. The method of claim 8 wherein said hybrid maize cell is the result of a cross between a first parent which is a derivative of stiff stalk and a second parent.

10. The method of claim 8 wherein said first parent is the male parent.

11. The method of claim 1 wherein said first parent is the female parent.
